(19) United States
(12) Patent Application Publication
(10) Pub. No.: US 2022/0243256 A1
(43) Pub. Date: Aug. 4, 2022

(54) Thermocycling PCR chip made of transparent graphene conductive film

(71) Applicants: Guozhi Zhu, Frederick, MD (US); Miao Qiao, Frederick, MD (US); Bide Qiao, AnShan (CN)

(72) Inventors: Guozhi Zhu, Frederick, MD (US); Miao Qiao, Frederick, MD (US); Bide Qiao, AnShan (CN)

(21) Appl. No.: 17/622,352

(22) PCT Filed: Jul. 5, 2020
(86) PCT No.: PCT/US2020/040848

§ 371 Date: Dec. 23, 2021

Related U.S. Application Data
(60) Provisional application No. 62/873,332, filed on Jul. 12, 2019.

(51) Int. Cl. C12Q 1/686; B01L 3/00; B01L 7/00

(52) U.S. Cl. CPC C12Q 1/686; B01L 3/502715; B01L 2300/0819; B01L 2300/0663; B01L 7/52

(57) ABSTRACT

In the present invention, graphene Transparent Conductive Film (TCF) is integrated into a disposable PCR chip in which PCR reaction containers are sandwiched by two graphene TCFs. PCR is performed by thermocycling of the on-chip two graphene TCF simultaneously. A temperature sensor in the PCR chip is to measure the chip temperature in a real-time manner during PCR reaction, and to provide a feedback to control the thermocycling of the graphene TCF.

### INTRODUCTION

Indium tin oxide (ITO) is widely used for transparent conductive films (TCFs) as touch screen due to its outstanding electrical conductivity and transparency. The disadvantage of ITO includes, the need for the rare element which increases its cost, the poor mechanical properties which prevent it from being utilized in flexible, stretchable and bendable devices.

Graphene is two-dimensional, hexagonal lattice of carbon atoms. Graphene is about 100 times stronger than steel. It is nearly transparent and conducts electricity and heat very efficiently. In addition to its excellent electrical conductivity and transparency, graphene exhibits high flexibility. Its most applications include semiconductor, electronics, batteries, touch screen and composites.

Polymerase chain reaction (PCR) is a widely used method in molecular biology to make copies (amplify) of DNA nucleic acids. In PCR, copies of DNA are exponentially amplified to millions of times. PCR is now an indispensable technique used in medical laboratories for a variety of applications including pathogen detection and sequencing.

PCR relies on thermal cycling (thermocycling) of PCR reaction solution to amplify DNA. PCR reaction solution generally includes buffer, DNA polymerase, primer and dNTPs. Thermocycling is the process of repeated cycles of heating and cooling of PCR reaction solution, to permit DNA melting and enzyme-driven replication respectively. Traditional PCR machines commonly use heavy metallic heating block whose thermocycling is to drive the thermocycling of PCR reaction solution. In 2012, a convective flow PCR machine was reported in which the block is not thermocycling. The mechanism of convective flow PCR is to confine PCR reaction solution in a cylindrical container whose bottom temperature is higher than its top, so that the fluid circulates through the container by itself and automatically shuffle the PCR reaction solution from round to round. In convective flow PCR, the blocks on both of the bottom and top keep stable temperature.

Graphene has been employed in PCR in two ways: 1) graphene nano-flakes in PCR reaction solution; 2) graphene TCF as heater for convective flow PCR. Specific concentration of Graphene nano-flakes (12-60 ug/ml) in PCR reaction solution was found to enhance PCR specificity (Jia, et al, Small, 2012; 8: 2011-2015), which is proposed to attribute to excellent heat transfer property of graphene flakes. In 2014, Chung reported to use graphene TCF in convective flow PCR (“Convection-based realtime polymerase chain reaction (PCR) utilizing transparent graphene heaters”, Chung, et al. Sensors, 2014 IEEE). In Chung's design, graphene TCF is incorporated into a PCR chip to enable convective flow of PCR reaction solution. Graphene TCF causing a “circulating flow of the reaction solution by convection” is claimed in his patent (Chung, et al. U.S. Pat. No. 10,138,513 B2; 11/2018).

Dr. Chung's patent and his publication represent the most similar prior-art to the present invention. It should be emphasized that in Dr. Chung's design, graphene TCF is not thermocycling but keep at a stable temperature, and the temperature difference between two sides of the PCR chip functions as a driven force to induce convective flow PCR. In contrast, the present invention utilizes the thermocycling of on-chip graphene TCFs to drive PCR. In the present invention, two graphene TCFs which sandwich PCR reaction solution are thermocycling simultaneously.

### SUMMARY

The present invention provides a device for DNA amplification. The device is a PCR chip comprising a PCR reaction container and two graphene TCFs, wherein the two graphene TCFs sandwich the PCR reaction container. PCR is performed by thermocycling of on-chip graphene TCFs.

In one embodiment of the invention, the PCR chip further comprises a temperature sensor, and/or a sample injection port for introducing solution into chip, and/or a channel for receiving solution into a reaction container.

In a preferred exemplary embodiment, the PCR reaction container has sidewall made of plastic film.

In one embodiment of the invention, the PCR reaction solution comprises fluorescent compound, including but not limited to, SYBR green, fluorescent labeled probes and primers. In another aspect, detecting fluorescent signals quantifies the DNA.

In a preferred exemplary embodiment, PCR reaction solution comprises a colorimetric dye to monitor PCR amplification, wherein the dye is visually detectable. The colorimetric dye includes but not limited to a pH dye, a pyrophosphate indicator, or a magnesium ion indicator. In another aspect, detecting a color change of the dye quantifies the DNA.

### BRIEF DESCRIPTION OF THE DRAWINGS

The patent or application file contains at least one figure executed in color. Copies of this patent or patent application publication with color figures will be provided by the Office upon request and payment of the necessary fee.

FIG. 1A is a top view of an exemplary embodiment of a PCR chip of the present invention.

FIG. 1B is a cross sectional view of 1A along the dotted line.

FIGS. 2A and 2B are, exemplary illustration of a PCR chip of the present invention, before and after PCR reaction, respectively. The representative color is for illustration purpose only, and to mimic the real color change shown in Example 2 and Example 3.

FIG. 3A is the comparison of thermal profiles of a GeneChecker PCR chip on top of one graphene TCF (light gray) and between two graphene TCFs (gray). FIG. 3B is the comparison of thermal profiles of two graphene TCFs which sandwich a GeneChecker PCR chip (gray) and NGPCR plate (black), respectively.

FIG. 4 is experimental PCR data of an exemplary PCR chip of the present invention. 4A is the thermal profile of a complete PCR reaction. FIG. 4B is the post-PCR photograph of a portion of the chip. FIG. 4C is gel electrophoresis of the two reactions of 4B.

FIG. 5 is experimental PCR data of an exemplary PCR chip of the present invention, by employing faster temperature programming. FIG. 5A is the thermal profile of a complete PCR reaction. FIG. 5B is a typical thermal profile of a single PCR cycle. FIG. 5C is the photograph of the whole PCR chip prior to PCR. FIG. 5D is the photograph of the same PCR chip after PCR reaction. FIG. 5E is the gel electrophoresis of the PCR reactions of 5D.

### DETAIL DESCRIPTION

Exemplary embodiment of the present invention will be described in detail hereinafter with reference to the accompanying drawings. In the drawings, the size and position of each components is for the purpose of understanding and clarity. It will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the claims.

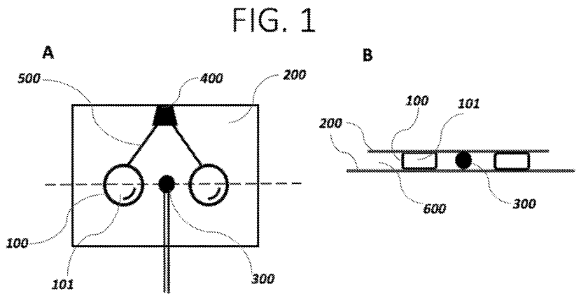

FIGS. 1A and 1B are top view and cross-sectional view illustrating the structure of the PCR chip representing an exemplary embodiment of the present invention.

Herein, the PCR chip comprises graphene TCFs 200, PCR reaction container 100 and PCR reaction solution 101. The two graphene TCFs 200 sandwich the PCR reaction container 100. Thermocycling of the two graphene TCFs 200 is to drive the PCR reaction of PCR reaction solution 101. In a preferred embodiment, the PCR reaction container 100 has sidewall made of thin plastic film. In one aspect, the top and bottom sidewall of PCR reaction container 100 is attached to graphene TCFs 200, by means such as, including but not limited to, heat-sealing, adhesive-sealing, radio-frequency sealing, light induced sealing. In another embodiment, the graphene TCFs 200 constitutes the top and bottom sidewall of PCR reaction container 100. The size of PCR reaction container 100 have a range, but not limited to, from 0.01 mm to 5 mm in diameter.

In a preferred embodiment, the PCR chip further comprise a temperature sensor 300, and/or a sample injection port 400, and/or a channel 500. Temperature sensor 300 is to sense the chip temperature. In a preferred embodiment, the temperature sensor 300 function to feedback the signal to control the thermocycling of graphene TCFs 200. Sample injection port 400 is to introduce fluid sample and PCR reaction solution into chip. Channel 500 is to receive fluid sample and PCR reaction solution into a reaction container. In a preferred embodiment, a PCR chip contains multiple PCR reaction containers 100, and multiple channels 500. PCR reaction solution 101 in each PCR reaction container 100 may amplify the same DNA. In addition, PCR reaction solution 101 in each PCR reaction container 100 may amplify different DNAs. In a preferred embodiment, injection port 400 and/or channel 500 comprises a valve or gate which is to prevent PCR reaction solution 101 in different PCR reaction containers 100 from mixing each other during PCR reaction.

The space 600 is between the two graphene TCFs 200 and surrounds the PCR reaction container 100. The space 600 may be intentionally reduced. In a preferred embodiment, the space 600 is reduced by sealing the two graphene TCFs 200 together. The two graphene TCFs 200 can be sealed or bonded or welded with a variety of means such as, including but not limited to, heat, adhesive, radio-frequency, light. In one embodiment, one graphene TCF 200 is more flexible and softer than the other graphene TCF 200, so that one graphene TCF 200 may “melt-down” to cover all of the top and surrounding surface of 100, 300, 400 and 500. The space 600 may be minimized in this scenario. In one aspect, at some location there is only one graphene TCF 200 exist, then the space 600 does not exist at that location.

In one embodiment, PCR reaction container 100 may contain some component of PCR reaction solution 101 prior to injection. In a preferred embodiment, PCR reaction container 100 may contain lyophilized component of PCR reaction solution 101.

The temperature sensor 300 is in a variety of forms such as, including but not limited to, thermocouple, thermopile, RTD, thermistor. In a preferred embodiment, the temperature sensor is sandwiched by the two graphene TCFs 200. The temperature sensor 300 may be outside of the PCR chip and sense the chip temperature in a non-contact manner. The temperature 300 may be an infrared sensor.

Light signal from PCR reaction solution 101 during PCR reaction may be, but not limited to, fluorescent or colorimetric. Taking advantage of the transparency of graphene TCFs 200, the light signal from PCR reaction solution 101 pass through the graphene TCFs 200. The light signal can be detected in real time manner during the amplification process, or at the endpoint of amplification. Real time detection of signal, especially by instruments, can quantify the amount of template DNA, and thus allows quantitative detection of DNA.

In one embodiment, the signal from PCR reaction solution 101 is fluorescent. Fluorescent compound is included in PCR reaction solution 101. The fluorescent compound includes but not limited to, SYBR, fluorescent labeled probes or primers.

In another embodiment, the light signal from PCR reaction solution 101 is colorimetric. In one aspect, a colorimetric dye is included in PCR reaction solution 101. The colorimetric dye is a visually detected colored dye. Its color is detectable in visible light under normal working environment. Colorimetric dyes such as pH dye, pyrophosphate indicator, magnesium ion dye have been used in DNA amplification. In one embodiment, the colorimetric dye includes but not limited to a pH dye, a pyrophosphate indicator, or a magnesium ion indicator.

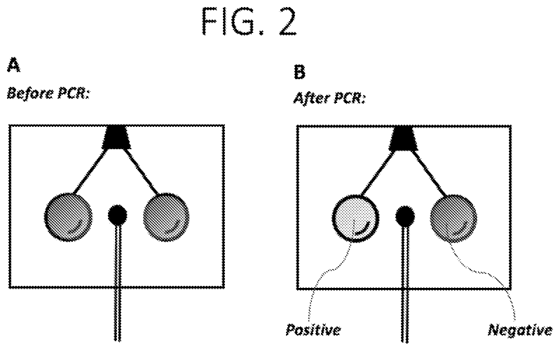

The color of dye can be detected and monitored by many means. Examples include, but are not limited to, the eyes of the operator, a colorimeter, a smartphone, a camera, a video recorder or a spectrophotometer. The term “detect” may be used interchangeably with the term “monitor” and “sense”. In exemplary embodiments demonstrated in Example 2 and Example 3, a colorimetric dye was used and the color change was detected by naked eyes. FIG. 2 mimics the color change and illustrates the application of colorimetric dye in the PCR chip for a visual detection of PCR. Embodiments of the invention provide a simple means for visual detection of nucleic acid amplification in PCR.

Graphene TCF has become commercial available and the price has dropped dramatically due to its large scaled production capacity these years. The cost of PCR chip in the present invention thus reduce and it is suitable to be disposable. In an exemplary embodiment, a PCR chip was designed to be in small size and with fast speed. The combined advantages of disposability, portability and fast speed enables the PCR chip of present invention to be very suitable for diagnostic applications in point-of-care testing or resource-limiting environment.

### EXAMPLES

### Example 1

### Thermocycling of Graphene TCF and Choice of PCR Reaction Container

Commercial graphene TCFs were purchased from Changzhou Erwei Tansu Technology Co. (catalog number JR029-H). The graphene TCFs has 120×141 mm in size, 0.25 mm in thickness and 2.4Ω electrical resistance. It is transparent and has 83% efficiency from electricity to heat.

Two different commercial PCR reaction containers were chosen and compared in this experiment: GeneChecker PCR chip and NGPCR PCR plate. GeneChecker Rapi:chip PCR chip is made of transparent polymer, and has 38 mm×25 mm×6 mm in size. Each chip has 10 containers. Each container has 0.5 mm in height and 8 mm long and 2 mm wide. Bottom of the chip is made of thin film for efficient heat transfer. With its specific PCR machine (GeneChecker UF-100 ultra-fast thermal cycler), the Rapi:chip finishes a 40 cycles PCR reaction in 20 minutes.

NGPCR 96-container PCR plate from MBS of Netherlands are formed of very thin polypropylene film, and has a volume of Sul for each container. The thin sidewall of the container ensures ultra-fast heat transfer and enables a 30 cycles PCR reaction in 2 minutes when run in its NEXTGENPCR 1 system.

A microcontroller (Arduino UNO) is employed to turn on/off the power to graphene TCF. A 19.5V DC was employed to power the graphene TCF through a relay controlled by a microcontroller. The temperature of graphene TCF was determined by time duration to power it. For example, at 22° C. ambient temperature, 2 overlapped JR029-H films connected in series can reach a stable 94° C. by repetition of 0.350 seconds power-on and 0.525 seconds power-off. A stable 70° C. is achieved by repetition of 0.225 seconds power-on and 0.760 seconds power-off. The heating time necessary for JR029-H film from 70° C. to 94° C. and cooling time from 94° C. to 70° C. were then counted respectively. All of these factors and thermocycling program settings were then incorporated into Arduino program code. The thermocycling program settings are 94° C. pre-denaturation for 2 min followed by 35 cycles of PCR amplification (94° C. for 5 seconds, and 70° C. for 7 seconds).

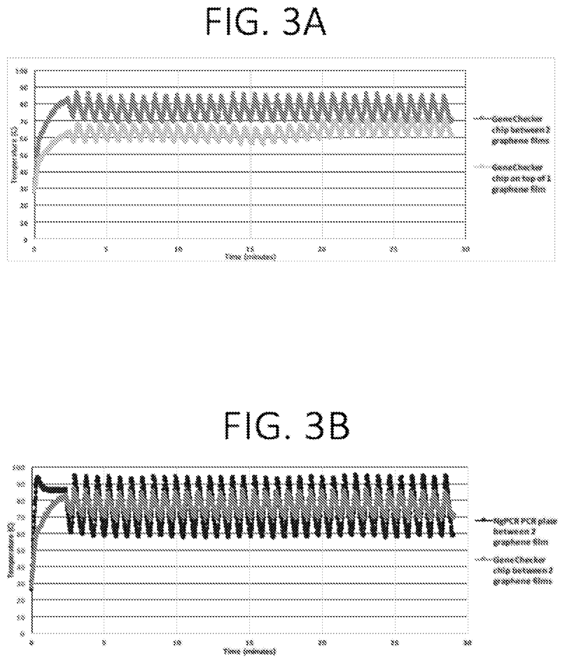

A K type thermocouple was linked to the microcontroller through a AD595 thermocouple amplifier. The thermocouple probe was insert into a container of a GeneChecker Rapi:chip. Firstly, the Rapi:chip was put on the surface of one graphene TCF and run the PCR program. The thermal profile of the complete PCR was obtained. As shown in FIG. 3A, its thermal profile (light gray) was in the range of 58° C. to 70° C. The 70° C. is not high enough to denature DNA, which is not satisfactory for a PCR reaction.

Then, two graphene TCFs were linked in series and overlapped to sandwich the GeneChecker Rapi:chip. The four edges (6.5 mm in height) of the chip was trimmed so that the top surface of the chip has a good contact with its above graphene TCF. The sandwich was tightly fastened by a clamp to prevent heat loss during PCR, and the same PCR program was run. The thermal profile (gray) was shown in FIG. 3A. As shown, the thermocycling of the Rapi:chip was in the range of 70° C. to 90° C., which is better than single graphene TCF. Therefore, two graphene TCFs in a sandwich format are chosen for the following examples. However, the temperature profile is still lower than what is needed for a PCR reaction. The reason for the chip to fail to reach to 94° C. probably is probably because the GeneChecker Rapi:chip has a thick top layer which prevent the heat transfer.

NGPCR plate has superb heat transfer ability because of its thin-sidewall. It was then tested to see whether a better thermocycling pattern can be achieved. A portion of a heat-sealed plate was cut off and a thermocouple probe was inserted into one container. Two graphene TCFs sandwiched the plate and the same PCR program of FIG. 3A was run. As shown in FIG. 3B, much better thermal profile (black) was obtained. The pre-denature step shows a stabilized temperature, and the thermocycling has a range from 60° C. to 95° C. Therefore, the container of NGPCR PCR plate is much better than GeneChecker Rapi:chip. NGPCR PCR plate achieved a better thermocycling pattern. According to the result of Example 1, satisfactory thermocycling is achieved for graphene TCFs by sandwiching thin-sidewall PCR reaction containers.

### Example 2

### PCR Reactions in Thin-Sidewall Containers Sandwiched by Two Thermocycling Graphene TCFs

PCR reactions were tested in an exemplary PCR chip of the present invention. A commercial PCR kit from GM Biosciences (Catalog: GM7099) for testing GFP tag was employed to prepare PCR reaction solution following the kit instruction. The PCR reaction solution contains a colorimetric dye to monitoring PCR amplification. The dye is visually detectable colored dye which change its original violet-purple color to blue upon PCR amplification. Naked eye can easily discriminate the color change. Taking fully advantage of the transparency of graphene films, PCR amplification can be directly monitored by naked eyes along the PCR reaction.

An exemplary PCR chip was prepared as followed. Two graphene TCFs (JR029-H) were connected in series and overlapped each other to sandwich a portion of NGPCR plate. Sul PCR reaction solution with/without 700 pg template (MigR1 plasmid) were loaded into two containers of NGPCR plate and heat sealed thereafter. The two containers were then placed between two graphene TCFs. A K type thermocouple was inserted into the 2 graphene TCFs at a location close to the NGPCR containers (within 5 centimeters distance) to monitor the chip temperature. Thus, the exemplary PCR chip has 2 graphene TCFs which sandwich PCR reaction containers and a temperature sensor.

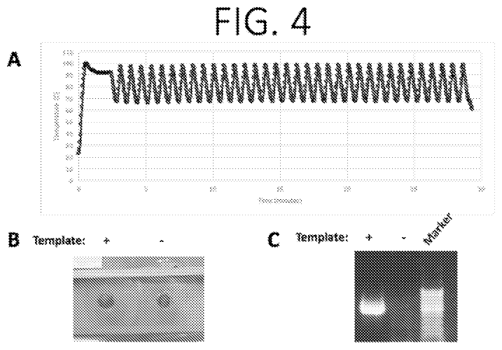

Thermocycling of the two graphene TCFs was achieved by a microcontroller with the program setting: 97° C. pre-denaturation for 2 min followed by 35 cycles of PCR amplification (97° C. for 5 seconds, and 73° C. for 7 seconds). The chip temperature was recorded during the PCR reactions. As shown in FIG. 4A, an excellent thermal profile of the PCR chip was achieved during PCR. It was observed that the container with template started to change its color at 25 cycles. A 35 cycles PCR reaction was finished in about 28 minutes. After the PCR completion, the chip was pictured (FIG. 4B) and subjected to gel electrophoresis (FIG. 4C). The result of FIG. 4B shows the expected color change of the containers in the chip. The container with template (left) turned to sky-blue color and the non-template container (right) kept its original violet-purple color. To further confirm the PCR reactions of the two containers in FIG. 4B, the two PCR products were subjected to agarose gel electrophoresis. As shown in FIG. 4C, a desired 500 bp amplification band was obtained for positive reaction but not for negative reaction. The agarose gel electrophoresis verified that the container with blue color had specific amplification, whereas the container with purple color did not have specific amplification. According to the result of Example 2, successful PCR reactions were achieved in an exemplary PCR chip by thermocycling of on-chip graphene TCFs.

### Example 3

### Fast PCR Reactions in a PCR Chip by Thermocycling On-Chip Graphene TCFs

Portability and speed are critical considerations for point-of-care application. Therefore, a smaller PCR chip was made and a variety of conditions were tested and tried to speed up PCR. The experiment below represents a typical exemplary embodiment of the present invention in which a very fast PCR by a small PCR chip was successfully achieved.

In this example, three modifications were employed to achieve portability and fast speed: 1) use small size of graphene TCFs; 2) use cooling fan to make cooling speed faster, and 3) feedback the chip temperature to microcontroller for automatic control of heating and cooling.

A new graphene TCF (JR029-E) was chosen. JR029-E has a size of 120×141 mm in size, 0.25 mm thickness and 5.6 SI electrical resistance. It is transparent and has 83% efficiency from electricity to heat. A small piece (120×34 mm) was cut from the JR029-E film, which measured a 35 n electrical resistance. Folded in half to attach the two ends of the graphene film together, and stapled its edges to give a smaller size of 58×34 mm. Two PCR reaction containers containing PCR reaction solution as prepared in Example 2 (one container with template, the other without template) were sandwiched between the folded graphene film.

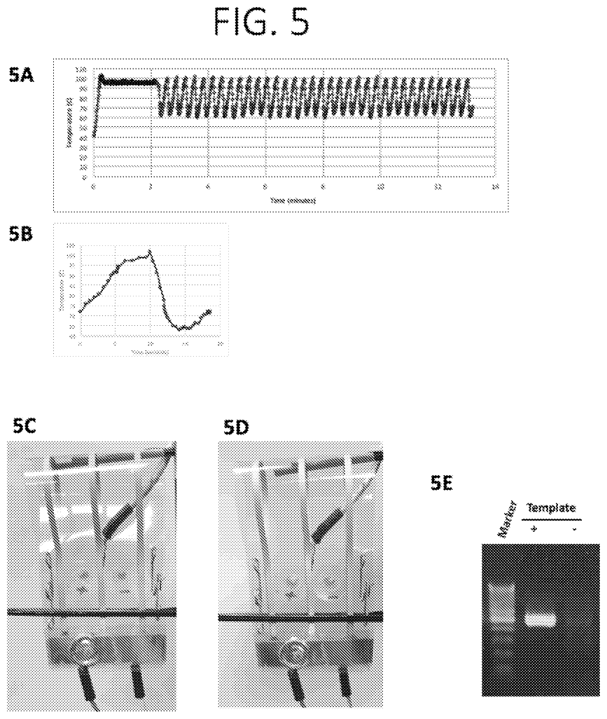

A K type thermocouple was inserted into the graphene films and in the middle of the two containers. Photograph of the whole PCR chip was taken as shown in FIG. 5C. It is noted that the original color of the two containers were violet-purple color before starting PCR reaction.

A 12V CPU cooling fan was connected to the microcontroller through a relay. It was positioned at the one flank of the PCR chip, so that the surface of both sides (front and rear) of PCR chip obtains sufficient air circulation.

Both the PCR chip and the cooling fan is automatically controlled by the microcontroller. When heating of the PCR chip, the graphene film is powered on and the cooling fan is powered off. When cooling of the PCR chip, the graphene film is powered off and the cooling fan is powered on. Based on the chip temperature sensed in a real-time manner, the microcontroller is programed in such a way that it automatically determines the time/duration to turn on/off the power to graphene TCF, and the time/duration to turn on/off the cooling fan. The automatic temperature programming not only accurately controls temperature but also shortens the reaction time.

The microcontroller was loaded with PCR program settings: 94° C. for 2 min followed by 35 cycles of 94° C. for 4 seconds and 70° C. for 7 seconds. The thermal profile of the PCR chip while running the PCR reaction was shown in FIG. 5A. The whole PCR reaction only took about 13 minutes. A detail thermal profiling is shown in FIG. 5B. Each cycle took an average of 19.0 seconds. Of the 19.0 seconds, heating the chip from 70° C. to 95° C. took only 5.2 seconds, and cooling from 95° C. to 70° C. took only 2.7 seconds (FIG. 5B). The result of FIGS. 5A and 5B demonstrated that fast thermocycling of the PCR chip of the present invention were successfully achieved.

After PCR completion, the photograph of the PCR chip was shown in FIG. 5D. The container with template (left) changed to blue color whereas the container without template (right) kept violet-purple color. The PCR product of the two containers shown in FIG. 5D was then subjected to gel electrophoresis and the result was shown in FIG. 5E. The container with color change has desired 500 bp band. According to the result of Example 3, very fast PCR reactions were achieved in an exemplary PCR chip by thermocycling on-chip graphene TCFs.

We claim:

1. A PCR chip for amplification of nucleic acid, comprising:
a reaction container which includes PCR reaction solution; and
two graphene Transparent Conductive Films (TCF) which sandwich the reaction container, wherein the temperature programming of the graphene TCFs enables the thermocycling of the PCR chip.

2. The PCR chip of claim 1 further comprising:
a sample injection port for introducing fluid sample and PCR reaction solution into chip;
and/or a channel for receiving fluid sample and PCR reaction solution into a reaction container;
and/or a temperature sensor for sensing the chip temperature.

3. A PCR chip according to claim 1, wherein the reaction container has sidewall made of plastic film.

4. A PCR chip according to claim 1, wherein the PCR reaction solution further comprising fluorescent compound, including but not limited to, SYBR, fluorescent labeled probes or primers.

5. A PCR chip according to claim 1, wherein the PCR reaction solution further comprising colorimetric dye.

6. A PCR chip according to claim 5, the colorimetric dye includes but not limited to a pH dye, a pyrophosphate indicator, or a magnesium ion indicator.

7. A PCR chip according to claim 5, wherein the colorimetric dye is detectable by means, including but not limited to, the eyes of the operator, a colorimeter, a smartphone, a camera, a video recorder or a spectrophotometer.

8. A PCR chip for amplification of nucleic acid, comprising:
a reaction container which include PCR reaction solution, wherein the solution contains visually detectable colorimetric dye; and
two graphene Transparent Conductive Films (TCF) which sandwich the reaction container, wherein the temperature programming of the graphene TCFs enables the thermocycling of the PCR chip.

9. The PCR chip of claim 8 further comprising:
a sample injection port for introducing fluid sample and PCR reaction solution into chip; and/or
a channel for receiving fluid sample and PCR reaction solution into a reaction container; and/or
a temperature sensor for sensing the chip temperature

10. A PCR chip according to claim 8, wherein the reaction container has sidewall made of plastic film.

11. A PCR chip according to claim 8, the colorimetric dye includes but not limited to a pH dye, a pyrophosphate indicator, or a magnesium ion indicator.

12. A PCR chip according to claim 8, wherein the colorimetric dye is detectable by means, including but not limited to, the eyes of the operator, a colorimeter, a smartphone, a camera, a video recorder or a spectrophotometer.
